(19) United States
(12) Patent Application Publication
(10) Pub. No.: US 2025/0025024 A1
(43) Pub. Date: Jan. 23, 2025

(54) MEDICAL SCOPE DEVICE STEERING CONTROLS

(71) Applicant: Cook Medical Technologies LLC, Bloomington, IN (US)

(72) Inventors: Diarmuid McGee, Galway (IE); Sorcha Ni Fhrighil, Limerick (IE); Stephen Hale, Lismore (IE); Ahmed Wanas, Limerick (IE)

(73) Assignee: Cook Medical Technologies LLC, Bloomington, IN (US)

(21) Appl. No.: 18/768,658

(22) Filed: Jul. 10, 2024

Related U.S. Application Data
(60) Provisional application No. 63/514,921, filed on Jul. 21, 2023.

(51) Int. Cl. A61B 1/005; A61B 1/00; A61B 1/012; A61B 1/045; A61B 1/273

(52) U.S. Cl. CPC A61B 1/0052; A61B 1/00029; A61B 1/0004; A61B 1/00042; A61B 1/00043; A61B 1/00128; A61B 1/0055; A61B 1/0057; A61B 1/0125; A61B 1/045; A61B 1/273

(57) ABSTRACT

A medical scope device may have a handle located at a proximal end of the medical scope device. The medical device may have a first steerable device extending from the handle to a distal end of the medical scope device and a second steerable device extending from the handle to the distal end of the medical scope device. The medical device may have a first control assembly disposed at a first location on the handle and a second control assembly disposed at a second location on the handle, offset from the first control assembly. The first control assembly may control a movement of a distal end of the first steerable device, and the second control assembly may control a movement of a distal end of the second steerable device.

### RELATED APPLICATIONS

This application claims the benefit of the filing date under 35 U.S.C. § 119 (e) of Provisional U.S. Patent Application Ser. No. 63/514,921, filed Jul. 21, 2023, which is hereby incorporated by reference.

### BACKGROUND

The statements in this section merely provide background information related to the present disclosure and may not constitute prior art.

The traditional endoscope is a medical device used in a variety of procedures. For example, a physician may insert the endoscope, for example, into a patient's mouth or into another body opening and then manipulate the distal end of the device through the patient's gastrointestinal (GI) tract to perform a particular endoscopic procedure. The physician may then use a variety of instruments during the procedure that are passed through an accessory channel that is located within the outer shaft of the endoscope. As the endoscopy field advances, new endoscopes are being created for specific procedures.

Endoscopes are typically steerable from a proximal-end handle. For example, certain steering features and mechanisms for controlling the distal end of the endoscope are discussed in U.S. Patent Application Pub. No. 2015/0366435, which is hereby incorporated by reference in its entirety. While steerable endoscopes are used with success to treat a variety of issues, existing endoscopes are often challenging to steer when inside the body. As such, the present disclosure presents an improved steering system for use with a variety of endoscopes to control the steering of multiple components.

### BRIEF DESCRIPTION OF THE DRAWINGS

In order that the present disclosure may be well understood, there will now be described various forms thereof, given by way of example, reference being made to the accompanying drawings. The components in the figures are not necessarily to scale. Moreover, in the figures, like-referenced numerals designate corresponding parts throughout the different views.

FIG. 1 is an illustration showing a steerable medical device in accordance with certain aspects of the present disclosure.

FIG. 2 is an illustration showing an embodiment of a handle for use with a steerable medical device in accordance with certain aspects of the present disclosure.

FIG. 3 is an illustration showing a portion of a steering system that controls two separate elongated steerable components in accordance with certain aspects of the present disclosure.

FIG. 4 is an illustration showing a portion of a steering system that controls two separate elongated steerable components in accordance with certain aspects of the present disclosure.

FIG. 5A is an illustration showing a set of control assemblies, where at least one of the control assemblies is engageable by a brake lever in accordance with certain aspects of the present disclosure.

FIG. 5B is an illustration showing a second view of certain features depicted in FIG. 5A, including two control assemblies and the first brake lever.

FIG. 5C is an illustration showing a brake lever for engaging at least one control assembly in accordance with certain aspects of the present disclosure.

FIG. 5D is an illustration showing a braking surface forming a wedge for coupling with the brake lever of FIG. 5C in accordance with certain aspects of the present disclosure.

FIG. 6A is an illustration showing a set of control assemblies, where at least one of the control assemblies is engageable by a brake dial in accordance with certain aspects of the present disclosure.

FIG. 6B is an illustration showing a second view of certain features depicted in FIG. 14, including two control assemblies and the brake dial.

FIG. 6C is an illustration showing the brake dial from FIGS. 6A-6B in isolation.

FIG. 6D is an illustration showing a control assembly with surface features that are configured for engagement with a brake dial in accordance with certain aspects of the present disclosure.

FIG. 7 is an illustration showing an electromechanical diagram for operating certain embodiments with the use of electronically-controlled motors (or other electromechanical actuators), potentially based on information received from a feedback loop, in accordance with certain aspects of the present disclosure.

### DETAILED DESCRIPTION

The following description is merely exemplary in nature and is not intended to limit the present disclosure, application, or uses. It should be understood that throughout the drawings, corresponding reference numerals indicate like or corresponding parts and features.

In adding reference denotations to elements of each drawing, although the same elements are displayed on a different drawing, it should be noted that the same elements have the same denotations. In addition, in describing one aspect of the present disclosure, if it is determined that a detailed description of related well-known configurations or functions blurs the gist of one aspect of the present disclosure, it will be omitted.

In the following discussion, the terms “proximal” and “distal” will be used to describe the opposing axial ends of the device, as well as the axial ends of various component features. The term “proximal” is used in its conventional sense to refer to the end of the device (or component) that is closest to the medical professional during use of the assembly. The term “distal” is used in its conventional sense to refer to the end of the device (or component) that is initially inserted into the patient, or that is closest to the patient during use. The term “longitudinal” will be used to refer to an axial direction that aligns with the proximal-distal axis of the device (or component), for example, when the device is not bent. The terms “radially” and “radial” will be used to refer to elements, surfaces, or assemblies relative to one another that may extend perpendicularly from a longitudinal axis. The terms “circumference,” “circumferentially,” and “circumferential” will be used to elements, surfaces, or assemblies relative to one another encircling or substantially encircling a longitudinal axis at a radius.

The uses of the terms “a” and “an” and “the” and similar references in the context of describing the present disclosure (especially in the context of the following claims) are to be construed to cover both the singular and the plural, unless otherwise indicated herein or clearly contradicted by context. The use of the term “plurality of” is defined by the Applicant in the broadest sense, superseding any other implied definitions or limitations hereinbefore or hereinafter unless expressly asserted by the Applicant to the contrary, to mean a quantity of more than one. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

As used herein, the terms “comprise(s),” “include(s),” “having,” “has,” “can,” “contain(s),” and variants thereof, are intended to be open-ended transitional phrases, terms, or words that do not preclude the possibility of additional acts or structures. The present description also contemplates other examples “comprising,” “consisting of,” and “consisting essentially of” the elements presented herein, whether explicitly set forth or not.

In describing elements of the present disclosure, the terms 1st, 2nd, first, second, A, B, (a), (b), and the like, may be used herein. These terms are only used to distinguish one element from another element, but do not limit the corresponding elements, irrespective of the nature or order of the corresponding elements.

Unless otherwise defined, all terms used herein, including technical or scientific terms, have the same meanings as those generally understood by those skilled in the art to which the present disclosure pertains. Such terms as those defined in a generally used dictionary are to be interpreted as having meanings equal to the contextual meanings in the relevant field of art.

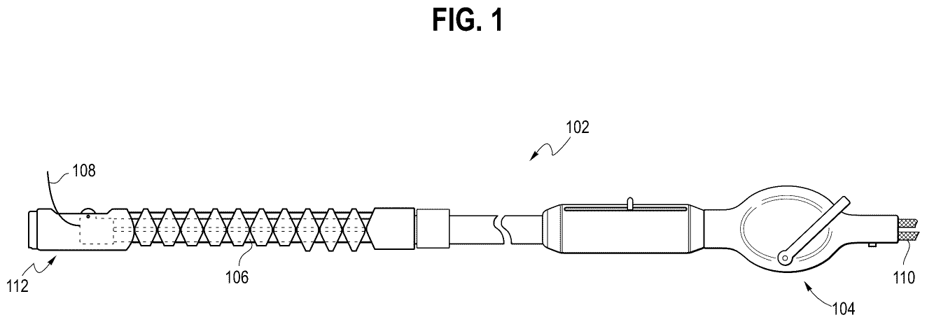

FIG. 1 shows an embodiment of a steerable medical device. The steerable medical device 102 includes a control handle 104 with a steerable outer sheath 106 extending distally therefrom. The outer sheath 106, which may also be referred to as an exoskeleton of the steerable medical device 102, may comprise an elongate tube extending from a distal end of the handle 104 to a distal end of the medical device 102. Without limitation, certain aspects of the steerable medical device 102 may include features discussed in U.S. patent application Ser. No. 16/749,083 (and corresponding Pub. No. 2020/0163534A1), which is hereby incorporated by reference in its entirety. For example, the steerable medical device 102 may generally include an endoscope (of any suitable type), and may include one or more accessory channels 110. The accessory channels may provide lumens or other pathways to a distal end 112 of the device 102 such that accessory components may extend to the device's distal end for performance of desired functions. For example, and without limitation, the accessory channels 110 may receive a device 108, which may be referred to as an accessory device, for example a cholangioscope, which is a relatively small-diameter device that is best delivered to a target site via a larger catheter (e.g., the outer sheath 106 and/or an accessory channel).

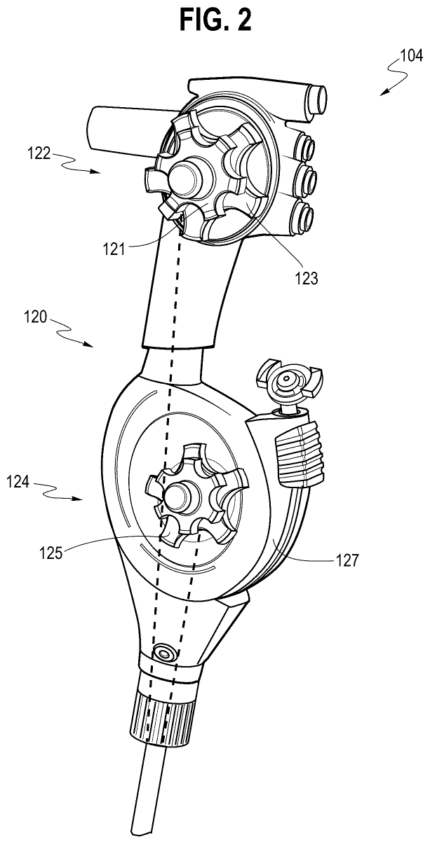

The handle 104 of the device 102 may be located at a proximal end of the steerable medical device 102, and therefore accessible directly by a medical professional. The handle may have any suitable features for steering the device, hereafter referred to as a steering control system 120. For example, an alternative handle 104 is shown in FIG. 2. Referring to FIG. 2, the steering control system 120 of the device includes a set of control assemblies, in particular a first control assembly 122 and a second control assembly 124. The first control assembly 122 and the second control assembly 124 may be configured to control any suitable feature of the device, such as left-right (L/R) and up-down motion (U/D) of the outer sheath 106 of the steerable medical device 102 and/or the device 108 extending through the accessory channel 110. For example, the first control assembly 122 may control the L/R and U/D movement of the outer sheath 106 and the second control assembly 124 may control the L/R and U/D movement of the device 108 received in the accessory channel 110. Each control assembly may include one or more wheels to control movement of a component of the device 102. For example, the first control assembly 122 may include a first L/R wheel 121 to control the left/right movement of the outer sheath 106 and/or a first U/D wheel 123 to control the up/down movement of the outer sheath 106. Additionally or alternatively, the second control assembly may include a second L/R wheel 125 to control the left/right movement of the device 108 in the accessory channel 110 an/or a second U/D wheel 127 to control the up/down movement of the device 108. Additionally or alternatively, the control assemblies 122, 124 may include additional or alternative control features other than wheels. For example, the control assemblies 122, 124 may utilize wheels, knobs, levers, or any other components or devices suitable to cause movement components of the device 102. Certain aspects of the handle 104 and the control assemblies may include features described U.S. patent application Ser. No. 15/655,239 (published as U.S. 2018/028778A1), entitled “STEERABLE MULTILUMEN CATHETER SHAFT,” which is hereby incorporated by reference in its entirety.

While the present disclosure generally describes the steering control system 120 as controlling (1) the outer sheath 106, and (2) a cholangioscope 108, those skilled in the art will recognize that the steering aspects discussed herein may apply to any suitable endoscopy device where steering ability is desirable, and particularly those components that are elongated such that steering is desirable at or near the distal end of an endoscope. For example, any of the following may be controlled via aspects discussed herein: accessory channels, cameras, graspers, forceps, needles, brushes, stents, dissectors, suture holders, balloons, extraction baskets, lithotripsy probes, laser fibers, and/or any other suitable endoscopy or other medical device. Also, the two steerable devices may also be moveable distally and/or proximally relative to each other, for example when an inner device advances distally relative to an outer sheath. Steering of either device may occur when one steerable device is advanced distally relative to the other, for example.

The steering system 120 may have the first control assembly 122 and the second control assembly 124 as its only steering actuators. Other actuator types are also contemplated as an alternative (or addition) to the control assemblies, such as switches, sliders, levers, one or more electronic controls (utilizing a joystick, motor, etc.) and the like. In the present embodiment, the first L/R wheel 121 and the first U/D wheel 123 of the may be coaxial rotatable relative to each other (and each rotatable relative to a handle or housing of the medical device 102), and generally adjacent to one another to facilitate one-hand use. Additionally or alternatively, the second L/R wheel 125 and the second U/D wheel 127 of the second control assembly 124 may be coaxially rotatable relative to each other and generally adjacent to one another to facilitate one-hand use. Advantageously, a physician or other medical professional may find this embodiment relatively easy to operate (perhaps with a single hand).

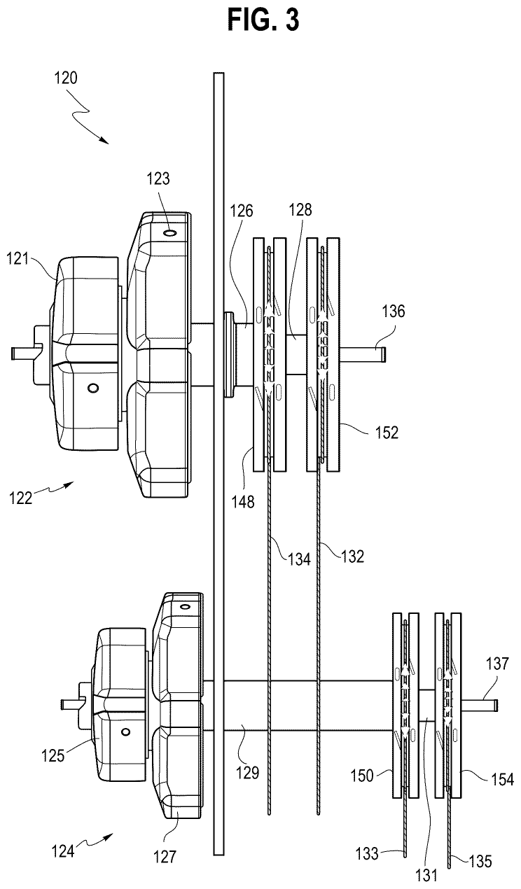

As shown in FIG. 3, a first U/D axle 126 may be fixed to the first U/D wheel 123 and a first L/R axle 128 may be fixed to the first L/R wheel 121. Each of the first U/D axle 126 and the first L/R axle 128 may extend inside the handle 104 of the device such that they are generally hidden from view. The first U/D axle 126 may be hollow such that the first L/R axle 128 may extend through the first U/D axle 126 in a co-axial manner. Since the first L/R axle 128 may need to be exposed within the handle of the device to reach a first L/R wire 132 (discussed below), the first L/R axle 128 may be longer than the first U/D axle 126 such that an end of the first L/R axle 128 within the handle of the device is exposed outside of the hollow first U/D axle 126. Optionally, the axles may be supported by a first central shaft 136 which is fixed relative to the handle's housing, and which may generally extend along the rotational axis of each of the first control assembly 122, the first U/D wheel 123, the first L/R wheel 121, the first U/D axle 126, and the first L/R axle 128.

A second U/D axle 129 may be fixed to the second U/D wheel 127 and a second L/R axle 131 may be fixed to the second L/R wheel 125. Each of the second U/D axle 129 and the second L/R axle 131 may extend inside the handle 104. The second U/D axle 129 may be hollow such that the second L/R axle 131 may extend through the second U/D axle 129 in a co-axial manner. Since the second L/R axle 131 may need to be exposed within the handle of the device to reach a second L/R wire 135 (discussed below), the second L/R axle 131 may be longer than the second U/D axle 129 such that an end of the second L/R axle 131 within the handle of the device is exposed outside of the hollow second U/D axle 129. Optionally, the axles may be supported by a second central shaft 137 which is fixed relative to the handle's housing, and which may generally extend along the rotational axis of each of the second control assembly 124, the second U/D wheel 127, the second L/R wheel 125, the second U/D axle 129, and the second L/R axle 131.

The first U/D axle 126 and the first L/R axle 128 may be mechanically coupled to drums. For example, referring to FIG. 3, the first U/D axle 126 may include a first U/D drum 148 (or other suitable connection element) that couples to a first L/R wire 132, and the second axle 128 may similarly include a first L/R drum 152 (or other suitable connection element) that couples to a first U/D control wire 134. Optionally, a motor may be utilized (e.g., connecting a steering actuator, which may be a motor switch), but this is optional.

Each of the drums 148, 152, 150, and 154 may include a groove that receives a pull wire, the pull wire extending to the distal end of the device for controlling certain distal-end movements. For example, the first U/D drum 148 and first L/R drum 152 may manipulate a first U/D control wire 134 and first U/D control wire 132, respectively, extending to a first steerable device, for example, the exoskeleton or outer sheath 106. The second U/D drum 150 and the second L/R drum 154 may manipulate a second U/D control wire 133 and second L/R control wire 135, respectively, extending to a second steerable device, for example, a device 108 in the accessory channel 110 such as a cholangioscope. In other embodiments, additional wire drums may be included (e.g., for additional steerable devices and/or for additional movement types).

In some embodiments, it may be advantageous to wrap a control wire around a wire drum such that it abuts about 180 degrees, or more, of the drum circumference, before anchoring it to the drum. This aspect prevents or substantially limits wire kinking when the drum rotates beyond 180 degrees (e.g., in a rotation direction that is opposite the direction the wire is initially wrapped around the drum). This aspect may be applicable to any of the above-described wire drums.

While any suitable arrangement is contemplated, the depicted embodiment has the first U/D drum 148 and first L/R drum 152 controlling the first steerable device, for example, the exoskeleton 106. For example, the first L/R drum 152 may control the position of the first steerable device in a first direction (optionally left-to-right), and the first U/D drum 148 may control the position of the first steerable device in a second direction (optionally up-and-down). The second U/D drum 150 and second L/R drum 154 may control the second steerable device 108 in a similar manner.

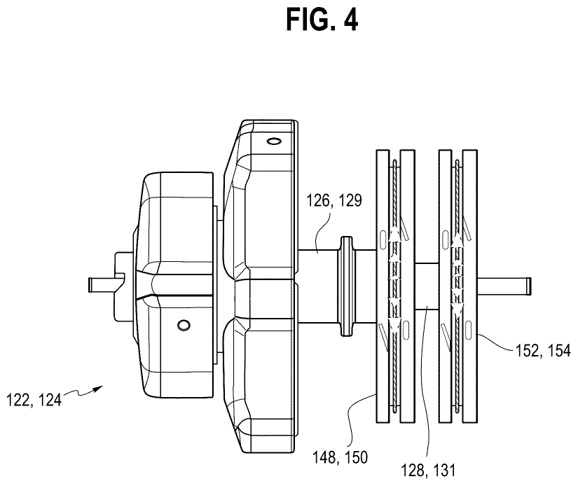

As shown in FIG. 4, the first and/or second U/D axle 126, 129 may include a sprocket (or other suitable connection element) that couples the first and/or second U/D drum 148, 150 the first U/D wire 134 or second U/D wire 133, respectively. Additionally or alternatively, and the first and/or second L/R axle 128, 131 may similarly include a sprocket (or other suitable connection element) that couples the first and/or second L/R drum 152, 154 to the first L/R wire 132 or second L/R wire 135, respectively. Additionally or alternatively, the first U/D wire 134, the second U/D wire 133, the first L/R wire 132, and/or second L/R wire 135 may be wires, but may alternatively be belts, ropes, strings, chains with a plurality of individual links, or any other suitable elongated cord or strand for connecting offset rotatable elements. Optionally, a motor may be utilized (e.g., connecting a steering actuator, which may be a motor switch), and the spline shaft, but this is optional.

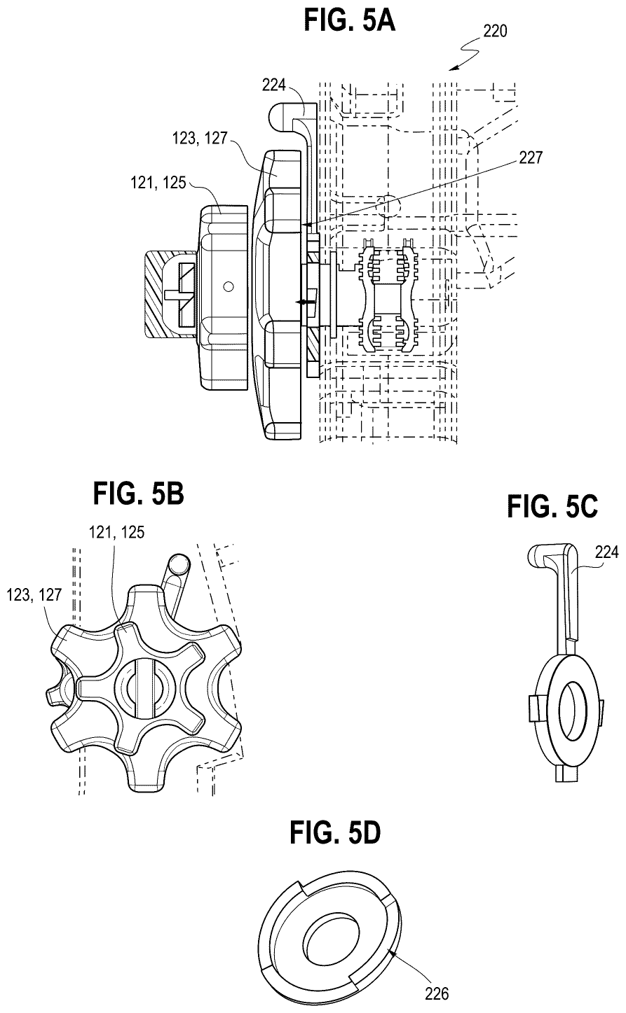

Any suitable brake may be included to prevent motion of the control assemblies 122, 124. For example, referring to FIGS. 5A-5D, a brake 220 may be included, which may engage the first U/D wheel 123 and/or the second U/D wheel 127 to prevent rotation of the first U/D wheel 123 and/or the second U/D wheel 127. The first brake 220 may have any suitable structure. For example, the brake may include a brake lever 224 that has an angled braking surface 226, where rotation of the brake lever 224 causes the angled braking surface 226 to act as a wedge against a corresponding brake surface 227 located between the first U/D wheel 123 and the brake lever 224 and/or the second U/D wheel 127 and the brake lever 224. As the brake lever 224 rotates further in a braking direction, the “wedge” aspect of the braking surface may increase static friction such that the first U/D wheel 123 and/or the second U/D wheel 127 cannot rotate, absent abnormal forces.

Additionally or alternatively, one or more of the brakes may be capable of providing variable resistance controlled by the physician, and in some embodiments a selected resistance level may be set and maintained by the device. For example, springs may be present such that once the brake position is determined for variable resistance, the spring resistance keeps the brake in such position even as the physician focuses on turning the steering controls (e.g., without worrying about also maintaining brake pressure). Other similar structures are also contemplated for adjusting and controlling brake force.

Optionally, a second brake 222 may be included, which may function to prevent rotation of the first L/R wheel 121 and/or the second L/R wheel 125. Advantageously, having separate brakes may allow the medical professional to selectively brake in desired directions, allowing motion in only a single direction in certain circumstances. The second brake 222 may include a brake dial 228, which may rotate coaxially with the U/D wheel 123 and the first L/R wheel 121 and/or the second U/D wheel 127 the second L/R wheel 125. The brake dial 228 may include one or more angled surfaces 229 that abut or otherwise engage a corresponding angled surface 231 located on, or adjacent to, the first L/R wheel 121 and/or the second L/R wheel 125. Thus, when the brake dial 228 rotates, it may axially move away from the first L/R wheel 121 and/or the second L/R wheel 125 such that it pulls a braking surface 223 into engagement with the first L/R wheel 121 and/or the second L/R wheel 125, fixing it from rotation. E.g., in this non-limiting example, the braking surface 223 may be located on a rod that is rigidly coupled with the brake dial 228, where the rod enters into the handle and passes axially through the control assemblies and terminates onto a braking pad that is inside the handle's shell. When the brake dial 227 is rotated, this rod may pull the brake into engagement with the L/R axle 128, 131 (which is fixed to the L/R wheel 121, 125), thereby creating a friction brake preventing movement of the first L/R wheel 121 and/or the second L/R wheel 125.

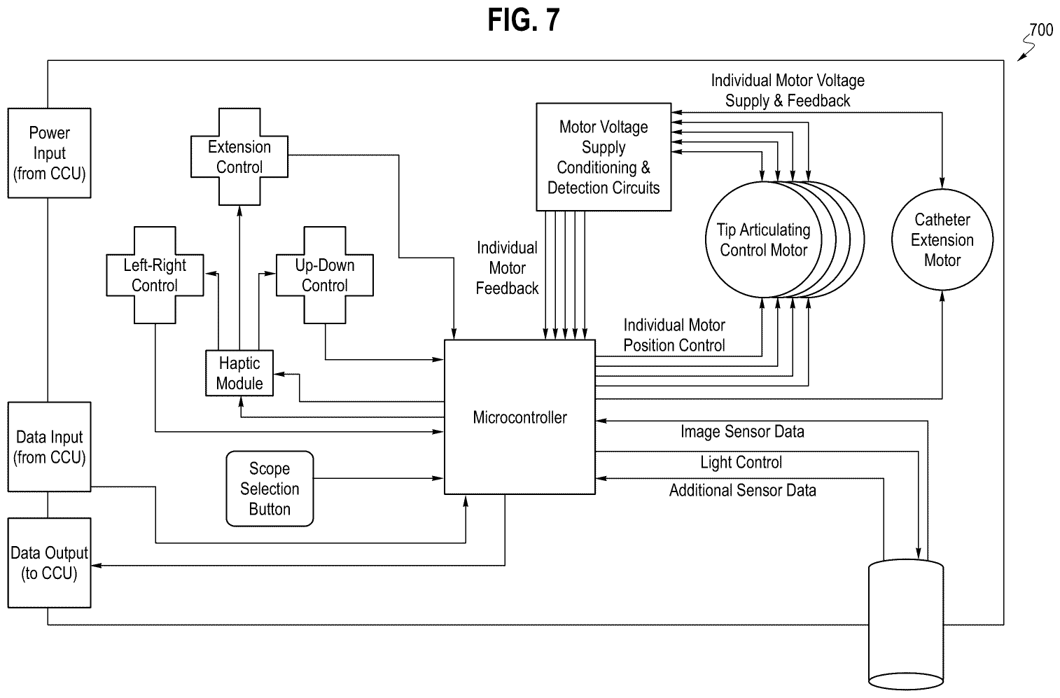

The aspects above may be controlled manually, where a human may directly control the wheels, fork selectors, and other components discussed above such that electronic control components are unnecessary. Other embodiments may include electromechanical aspects to automate certain motions, which may enhance the accuracy and precision of certain motions and reduce instances of human error. FIG. 7 shows an example of an electromechanical control system 700 for carrying out certain functions of a steering system similar to embodiments discussed above. For example, the electromechanical control system 700 may include one or more motors, which may include rotational or linear motor, servos, or other electromechanical modules for articulating the endoscope tip with the use of tension wires, strings or similar flexible material extending the endoscope length (from proximal to distal). When a tip articulation motor is included (as depicted) for generally steering the outer sheath of the device, the tip articulation motor may be shared, or one or more additional motors may be used for cholangioscope catheter, or another accessory, length extension from the distal tip.

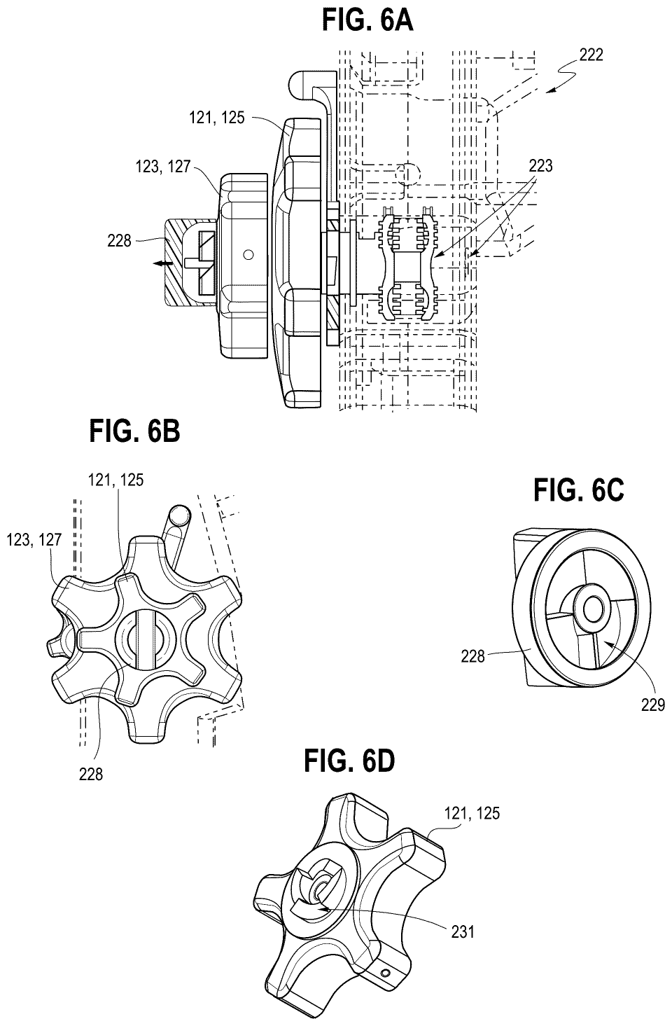

When included, the motors or other electronically-controlled actuators may be controlled by one or more microcontrollers located within the endoscope proximal handle assembly. Each motor (if there are multiple) may have an individual or common power supply, positional control, and/or motor status feedback (current, voltage, impedance, or other relevant electrical status). For example, using an encoder on the control assemblies, which may be similar to those discussed above with reference to FIGS. 1-6, to control a corresponding servomotor or other type of motor that is coupled to the respective drum. Thus, the control assembly and/or wheel is rotated and the motor rotates the drum by a corresponding amount. The left/right brake and up/down brake (e.g., brakes as discussed above with reference to FIGS. 5-6) can then also be read by an encoder that then communicates to the motor to either stop or decrease degrees rotation per amount of rotation on the control assembly and/or wheel. Advantageously, this motor power-control-feedback loop allows precise, accurate movement of each wire drum and can provide force, resistance, and/or tension feedback information for further processing, analysis, and use by the device microcontroller(s). The power-control feedback loop (with or without the microcontroller) may be embedded within each motor or contained within the proximal handle, along with an optional battery and/or other electronics. The motor(s) can be operated with or without the full power-control-feedback loop depending on component selection, desired level of control, and required status feedback for the rest of the system.

Position, via manipulation of the wire drums with the motors, may be controlled by turning the power on/off, switching power polarity, with a separate control signal, or a combination of these or any other suitable method. User control of motors may be provided via wheels, joysticks, buttons, or other user input devices located on the proximal handle assembly. A button, switch or other similar user input mechanism is provided for switching between the duodenoscope and cholangioscope. The user input device(s) can be either analog or digital with integrated electronics interpreting user input, raw electrical output such as variable resistors, or a combination of both or other similar implementations. Additionally, the user input device(s) and supporting electronics may be connected to the microcontroller(s), which may provide required filtering, interpretation, and transfer of user control input to motor control output.

Motor feedback described previously can also be used to provide haptic feedback to the user to simulate mechanical control and feedback via haptic modules included within the handle assembly. Haptic modules can include, but are not limited to, vibration, tension, force, torque. These haptic modules are controlled by the microcontroller(s) within the proximal handle assembly electronics, integrated within user input device(s) or a combination of both. Power is provided to the proximal handle assembly by a cable connection to the Camera Control Unit (CCU).

In addition to switching steering and/or fluid functionality, the embodiments herein may additionally or alternatively be capable of switching other functionalities between various components. For example, the embodiments herein may be capable of switching between cameras (e.g., where each steerable device has a camera), electromechanical sensors, actuatable electronic devices (e.g., brushes or the like), or any other suitable device(s).

Each component may include additional, different, or fewer components. For example, the handle 104 may have additional or fewer components, such as additional control assemblies, drums, and/or wires. Additionally or alternatively, the control assemblies and axles may have additional or fewer components, such as additional wheels, drums, and/or brakes.

The medical device 102 may be implemented with additional, different, or fewer components. For example, medical device 102 may have additional outer sheaths 106, accessory channels 110, and/or insertable devices 108.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed. Unless otherwise indicated or the context suggests otherwise, as used herein, “a” or “an” means “at least one” or “one or more.”

While various embodiments have been described, it will be apparent to those of ordinary skill in the art that many more embodiments and implementations are possible. Accordingly, the embodiments described herein are examples, not the only possible embodiments and implementations.

Having described various aspects of the subject matter above, additional disclosure is provided below that may be consistent with the claims originally filed with this disclosure. In describing this additional subject matter, reference may be made to the previously described figures. Any of the following aspects may be combined, where compatible.

The subject-matter of the disclosure may also relate, among others, to the following aspects:

A first aspect relates to a medical scope device, comprising: a handle located at a proximal end of the medical scope device; a first steerable device extending from the handle to a distal end of the medical scope device; a second steerable device extending from the handle to the distal end of the medical scope device; a first control assembly disposed at a first location on the handle; and a second control assembly disposed at a second location on the handle, wherein the first control assembly controls a movement of a distal end of the first steerable device, and wherein the second control assembly controls a movement of a distal end of the second steerable device.

A second aspect relates to the medical scope device of aspect 1, wherein an axis of rotation of the first control assembly is offset from an axis of rotation of the second control assembly.

A third aspect relates to the medical scope device of any preceding aspect, wherein the first control assembly is disposed closer to a proximal end of the handle than the second control assembly.

A fourth aspect relates to the medical scope device of any preceding aspect, wherein the first steerable device is an elongate tube extending from a distal end of the handle to the distal end of the medical scope device.

A fifth aspect relates to the medical scope device of any preceding aspect further comprising an accessory channel disposed within the elongate tube, the accessory channel extending along the elongate tube from the handle to the distal end of the medical scope device.

A sixth aspect relates to the medical scope device of any preceding aspect, wherein the second steerable device is disposed within the accessory channel, the second steerable device extending along the accessory channel from the handle to the distal end of the medical scope device.

A seventh aspect relates to the medical scope device of any preceding aspect, further comprising a first brake for selectively preventing rotation of the first control assembly and a second brake for selectively preventing rotation of the second control assembly.

An eighth aspect relates to the medical scope device of any preceding aspect, wherein the first control assembly comprises a first wheel and a second wheel, wherein the first wheel controls a first direction of movement of the first steerable device and the second wheel controls a second direction of movement of the first steerable device.

A ninth aspect relates to the medical scope device of any preceding aspect, wherein a first drum is coupled to the first wheel and wherein a second drum is coupled to the second wheel, wherein a first wire disposed on the first drum controls the first direction of movement of the first steerable device and a second wire disposed on the second drum controls the second direction of movement the first steerable device.

A tenth aspect relates to the medical scope device of any preceding aspect, wherein the second control assembly comprises a third wheel and a fourth wheel, wherein the third wheel controls a first direction of movement of the second steerable device and the fourth wheel controls a second direction of movement of the second steerable device.

An eleventh aspect relates to the medical scope device of any preceding aspect, wherein the first wheel and the second wheel of the first control assembly are coaxial, and wherein the third wheel and the fourth wheel of the second control assembly are coaxial.

A twelfth aspect relates to the medical scope device of any preceding aspect, wherein a third drum is coupled to the third wheel and wherein a fourth drum is coupled to the fourth wheel, wherein a third wire disposed on the third drum controls the first direction of movement of the second steerable device and a fourth wire disposed on the fourth drum controls the second direction of movement of the second steerable device.

A thirteenth aspect relates to a medical scope device, comprising: a handle at a proximal end of the medical scope device; an elongate tube extending from the handle to a distal end of the medical scope device; an accessory device disposed within the elongate tube and extending from the handle to the distal end of the medical scope device; a first control assembly disposed at a first location on the handle; and a second control assembly disposed at a second location on the handle offset from the first location; wherein the first control assembly controls a movement of the elongate tube, and wherein the first control assembly controls a movement of a distal end of the accessory device.

A fourteenth aspect relates to the medical scope device of any preceding aspect, wherein the accessory device is moveable distally relative to the elongate tube.

A fifteenth aspect relates to the medical scope device of any preceding aspect, further comprising a first brake for selectively preventing rotation of the first control assembly and a second brake for selectively preventing rotation of the second control assembly.

A sixteenth aspect relates to the medical scope device of any preceding aspect, wherein the first control assembly comprises a first wheel and a second wheel, wherein the first wheel controls a first direction of movement of the elongate tube and the second wheel controls a second direction of movement of the elongate tube.

A seventeenth aspect relates to the medical scope device of any preceding aspect, wherein a first drum is coupled to the first wheel and wherein a second drum is coupled to the second wheel, wherein a first wire disposed on the first drum controls the first direction of movement of the elongate tube and a second wire disposed on the second drum controls the second direction of movement of the elongate tube, the first drum and second drum coaxial with the first control assembly.

An eighteenth aspect relates to the medical scope device of any preceding aspect, wherein the second control assembly comprises a third wheel and a fourth wheel, wherein the third wheel controls a first direction of movement of the accessory device and the fourth wheel controls a second direction of movement of the accessory device.

A nineteenth aspect relates to the medical scope device of any preceding aspect, wherein a third drum is coupled to the third wheel and wherein a fourth drum is coupled to the fourth wheel, wherein a third wire disposed on the third drum controls the first direction of movement of the accessory device and a fourth wire disposed on the fourth drum controls the second direction of movement of the accessory device. the first drum and second drum coaxial with the first control assembly.

A twentieth aspect relates to a medical scope device, comprising: a handle located at a proximal end of the medical scope device; a first steerable device extending from the handle to a distal end of the medical scope device; a second steerable device extending from the handle to the distal end of the medical scope device; a first control assembly disposed at a first location on the handle, the first control assembly including a first wheel to control a first direction of movement of the first steerable device and a second wheel to control a second direction of movement of the first steerable device; and a second control assembly disposed at a second location on the handle offset from the first location, the second control assembly including a third wheel to control a first direction of movement of the second steerable device and a fourth wheel to control a second direction of movement of the second steerable device.

In addition to the features mentioned in each of the independent aspects enumerated above, some examples may show, alone or in combination, the optional features mentioned in the dependent aspects and/or as disclosed in the description above and shown in the figures.

1. A medical scope device, comprising:
a handle located at a proximal end of the medical scope device;
a first steerable device extending from the handle to a distal end of the medical scope device;
a second steerable device extending from the handle to the distal end of the medical scope device;
a first control assembly disposed at a first location on the handle; and
a second control assembly disposed at a second location on the handle,
wherein the first control assembly controls a movement of a distal end of the first steerable device, and
wherein the second control assembly controls a movement of a distal end of the second steerable device.

2. The medical scope device of claim 1, wherein an axis of rotation of the first control assembly is offset from an axis of rotation of the second control assembly.

3. The medical scope device of claim 2, wherein the first control assembly is disposed closer to a proximal end of the handle than the second control assembly.

4. The medical scope device of claim 1, wherein the first steerable device is an elongate tube extending from a distal end of the handle to the distal end of the medical scope device.

5. The medical scope device of claim 4 further comprising an accessory channel disposed within the elongate tube, the accessory channel extending along the elongate tube from the handle to the distal end of the medical scope device.

6. The medical scope device of claim 5, wherein the second steerable device is disposed within the accessory channel, the second steerable device extending along the accessory channel from the handle to the distal end of the medical scope device.

7. The medical scope device of claim 1, further comprising a first brake for selectively preventing rotation of the first control assembly and a second brake for selectively preventing rotation of the second control assembly.

8. The medical scope device of claim 1, wherein the first control assembly comprises a first wheel and a second wheel, wherein the first wheel controls a first direction of movement of the first steerable device and the second wheel controls a second direction of movement of the first steerable device.

9. The medical scope device of claim 8, wherein a first drum is coupled to the first wheel and wherein a second drum is coupled to the second wheel, wherein a first wire disposed on the first drum controls the first direction of movement of the first steerable device and a second wire disposed on the second drum controls the second direction of movement the first steerable device.

10. The medical scope device of claim 8, wherein the second control assembly comprises a third wheel and a fourth wheel, wherein the third wheel controls a first direction of movement of the second steerable device and the fourth wheel controls a second direction of movement of the second steerable device.

11. The medical scope device of claim 10, wherein the first wheel and the second wheel of the first control assembly are coaxial, and wherein the third wheel and the fourth wheel of the second control assembly are coaxial.

12. The medical scope device of claim 10, wherein a third drum is coupled to the third wheel and wherein a fourth drum is coupled to the fourth wheel, wherein a third wire disposed on the third drum controls the first direction of movement of the second steerable device and a fourth wire disposed on the fourth drum controls the second direction of movement of the second steerable device.

13. A medical scope device, comprising:
a handle at a proximal end of the medical scope device;
an elongate tube extending from the handle to a distal end of the medical scope device;
an accessory device disposed within the elongate tube and extending from the handle to the distal end of the medical scope device;
a first control assembly disposed at a first location on the handle; and
a second control assembly disposed at a second location on the handle offset from the first location;
wherein the first control assembly controls a movement of the elongate tube, and
wherein the second control assembly controls a movement of a distal end of the accessory device.

14. The medical scope device of claim 13, wherein the accessory device is moveable distally relative to the elongate tube.

15. The medical scope device of claim 13, further comprising a first brake for selectively preventing rotation of the first control assembly and a second brake for selectively preventing rotation of the second control assembly.

16. The medical scope device of claim 13, wherein the first control assembly comprises a first wheel and a second wheel, wherein the first wheel controls a first direction of movement of the elongate tube and the second wheel controls a second direction of movement of the elongate tube.

17. The medical scope device of claim 16, wherein a first drum is coupled to the first wheel and wherein a second drum is coupled to the second wheel, wherein a first wire disposed on the first drum controls the first direction of movement of the elongate tube and a second wire disposed on the second drum controls the second direction of movement of the elongate tube, the first drum and second drum coaxial with the first control assembly.

18. The medical scope device of claim 16, wherein the second control assembly comprises a third wheel and a fourth wheel, wherein the third wheel controls a first direction of movement of the accessory device and the fourth wheel controls a second direction of movement of the accessory device.

19. The medical scope device of claim 18, wherein a third drum is coupled to the third wheel and wherein a fourth drum is coupled to the fourth wheel, wherein a third wire disposed on the third drum controls the first direction of movement of the accessory device and a fourth wire disposed on the fourth drum controls the second direction of movement of the accessory device. the first drum and second drum coaxial with the first control assembly.

20. A medical scope device, comprising:
a handle located at a proximal end of the medical scope device;
a first steerable device extending from the handle to a distal end of the medical scope device;
a second steerable device extending from the handle to the distal end of the medical scope device;
a first control assembly disposed at a first location on the handle, the first control assembly including a first wheel to control a first direction of movement of the first steerable device and a second wheel to control a second direction of movement of the first steerable device; and
a second control assembly disposed at a second location on the handle offset from the first location, the second control assembly including a third wheel to control a first direction of movement of the second steerable device and a fourth wheel to control a second direction of movement of the second steerable device.
